(19) United States
(12) Patent Application Publication
(10) Pub. No.: US 2024/0082215 A1
(43) Pub. Date: Mar. 14, 2024

(54) USE OF MGLUR5 ANTAGONISTS FOR TREATING GAMBLING DISORDER

(71) Applicants: Novartis AG, Basel (CH); Yale University, New Haven, CT (US)

(72) Inventors: Fabrizio GASPARINI, Basel (CH); Baltazar GOMEZ-MANCILLA, Basel (CH); John KRYSTAL, New Haven, CT (US); Vincent MALATERRE, Basel (CH); Stephanie O'MALLEY, New Haven, CT (US); Godfrey PEARLSON, New Haven, CT (US); Marc POTENZA, New Haven, CT (US)

(73) Assignees: Novartis AG, Basel (CH); Yale University, New Haven, CT (US)

(21) Appl. No.: 18/266,917

(22) PCT Filed: Dec. 9, 2021
(86) PCT No.: PCT/IB2021/061506

§ 371 Date: Jun. 13, 2023

Related U.S. Application Data
(60) Provisional application No. 63/125,181, filed on Dec. 14, 2020.

(51) Int. Cl. A61K 31/404; A61P 25/30

(52) U.S. Cl. CPC A61K 31/404; A61P 25/30

(57) ABSTRACT

The present invention relates to the use of mavoglurant in the treatment of gambling disorder. The present invention also relates to the use of mavoglurant in the treatment of gaming disorder.

### STATEMENT REGARDING FEDERALLY FUNDED RESEARCH

This invention was made with government support under AA012870 awarded by National Institutes of Health. The government has certain rights in the invention.

The present invention relates to the use of mavoglurant in the treatment of gambling disorder. The present invention also relates to the use of mavoglurant in the treatment of gaming disorder.

### FIELD OF THE INVENTION

In one aspect the invention relates to the use of the mGluR5 antagonist named mavoglurant, or a pharmaceutically acceptable salt thereof, in the treatment of gambling disorder; in a treatment in the reduction of gambling by a gambling disorder patient; in a treatment to prevent relapse into gambling by a gambling disorder patient; in a treatment to promote gambling abstinence by a gambling disorder patient; in the treatment of the symptoms of depression or anxiety associated with gambling disorder.

In another aspect the invention relates to the use of the mGluR5 antagonist named mavoglurant, or a pharmaceutically acceptable salt thereof, in the treatment of gaming disorder; in a treatment in the reduction of gaming by a gaming disorder patient; in a treatment to prevent relapse into gaming by a gaming disorder patient; in a treatment to promote gaming abstinence by a gaming disorder patient; in the treatment of the symptoms of depression or anxiety associated with gaming disorder.

### BACKGROUND OF THE INVENTION

Gambling disorder, is a complex psychiatric disorder that has been defined with reference to DSM-5 criteria (i.e. according to the Diagnostic and Statistical Manual of Mental Disorders. 5^{th} Edition, Washington, DC: American Psychiatric Association, 2013). Gambling disorder is a significant worldwide health problem having adverse medical, social and economic effects (e.g. Potenza M N, Balodis I M, Derevensky J, Grant J E, Petry N M, Verdejo-Garcia A, W Y S. Gambling disorder. Nature Reviews Disease Primers. 2019; 5:51). To date, there is no medication approved by the US Food and Drug Administration (FDA) for use in the treatment of this disorder. Accordingly, there is a need to identify therapeutic agents that can be used to treat it, in particular drugs that are effective on reducing gambling cravings, promoting abstinence or reducing relapse once patients are abstinent.

Gaming disorder, also called internet gaming disorder, is a complex psychiatric disorder as well. Gaming disorder has been defined with reference to ] and research criteria for internet gaming disorder are defined in Section III in DSM-5. Gaming disorder is also a significant worldwide health problem having adverse medical, social and economic effects (King D L, Wölfling K, Potenza M N (2020) Taking gaming disorder treatment to the next level. JAMA Psychiatry 77(8):869-870). To date, there is no medication either approved by the US Food and Drug Administration (FDA) for use in the treatment of this disorder. Accordingly, there is also a need to identify therapeutic agents that can be used to treat it, in particular drugs that are effective on reducing gaming cravings, promoting abstinence or reducing relapse once patients are abstinent.

How people process rewards has been a central consideration in addictions. Multiple theories have been forwarded regarding reward processing abnormalities that may drive engagement in addictive behaviors. For example, the reward deficiency theory proposes that people with addictions are motivated to participate in addictive behaviors as their responses to natural rewards (e.g., palatable food) or non-addiction learned rewards (e.g., money) are relatively blunted (Blum K, Cull J G, Braverman E R, Comings D E. Reward deficiency syndrome. Am Scientist. 1996; 84:132-145). Positive reinforcement motivations (e.g., using substances or engaging in addictive behaviors for thrills or to achieve highs) and negative reinforcement motivations (e.g., using substances or engaging in addictive behaviors to relieve stress or escape from depression or other uncomfortable mood states) have also been incorporated into models of addiction and may also link to how people process rewards (e.g. Volkow N D, Koob G F, McLellan A T. Neurobiologic Advances from the Brain Disease Model of Addiction. The New England journal of medicine. 2016; 374:363-371; Brand M, Wegmann E, Stark R, Muller A, Wolfling K, Robbins T W, Potenza M N. The Interaction of Person-Affect-Cognition-Execution (I-PACE) model for addictive behaviors: Update, generalization to addictive behaviors beyond internet-use disorders, and specification of the process character of addictive behaviors. Neurosci Biobehav Rev. 2019; 104:1-10; Brand M, Young K, Laier C, Wölfling K, Potenza M N. Integrating psychological and neurobiological considerations regarding the development and maintenance of specific Internet-use disorders: An Interaction of Person-Affect-Cognition-Execution (I-PACE) model. Neurosci Biobehav Rev 2016; 71:252-266). One of the most widely used tasks to investigate neural correlates of reward processing is the monetary incentive delay task (MIDT). The MIDT was developed for use in humans based on animal research (Schultz W, Apicella P, Scarnati E, Ljungberg T. Neuronal activity in monkey ventral striatum related to the expectation of reward. J Neurosci. 1992; 12:4595-4610; Schultz W, Tremblay L, Hollerman J R. Reward processing in primate orbitofrontal cortex and basal ganglia. Cerebral Cortex. 2000; 10:272-284). The initial version for humans retained cues (e.g., triangles, squares) that signified conditions and could be used to investigate brain correlates of the processing of working for monetary reward or avoiding losses and anticipatory and consummatory (outcome) phases thereof [e.g. Knutson B, Fong G W, Adams C M, Varner J L, Hommer D. Dissociation of reward anticipation and outcome with event-related fMRI. Neuroreport. 2001; 12:3683-3687; Knutson B, Fong G W, Bennett S M, Adams C M, Hommer D. A region of mesial prefrontal cortex tracks monetarily rewarding outcomes: characterization with rapid event-related fMRI. Neuroimage. 2003; 18:263-272; Knutson B, Adams C M, Fong G W, Hommer D. Anticipation of increasing monetary reward selectively recruits nucleus accumbens. J Neurosci. 2001; 21:RC159 (151-155)]. The MIDT has been used in studies of addictions (e.g. Knutson B, Greer S M. Anticipatory affect: neural correlates and consequences for choice. Phil Trans Roy Soc B. 2008; 363:3771-3786; Balodis I M, Potenza M N. Anticipatory reward processing in addicted populations: a focus on the monetary incentive delay task. Biol Psychiatry 2015; 77:434-444; Luijten M, Schellekens A F, Kuhn S, Machielse M W, Sescousse G. Disruption of Reward Processing in Addiction: An Image-Based Meta-analysis of Functional Magnetic Resonance Imaging Studies. JAMA Psychiatry. 2017; 74:387-398). One widely replicable finding is that during reward anticipatory processing, individuals with addictions [to alcohol (e.g. Beck A, Schlagenhauf F, Wustenberg T, Hein J, Kienast T, Kahnt T, Schmack K, Hagele C, Knutson B, Heinz A, Wrase J. Ventral striatal activation during reward anticipation correlates with impulsivity in alcoholics. Biol Psychiatry. 2009;6 6:734-742; Wrase J, Schlagenhauf F, Kienast T, Wüstenberg T, Bermpohl F, Kahnt T, Beck A, Ströhle A, Juckel G, Knutson B, Heinz A. Dysfunction of reward processing correlates with alcohol craving in detoxified alcoholics. Neuroimage. 2007; 35:787-794), tobacco (e.g. Peters J, Bromberg U, Schneider S, Brassen S, Menz M, Banaschewski T, Conrod P J, Flor H, Gallinat J, Garavan H, Heinz A, Itterman B, Lathrop M, Martinot J L, Paus T, Poline J B, Robbins T W, Rietschel M, Smolka M, Ströhle A, Struve M, Loth E, Schumann G, Büchel C, Consortium. I. Lower ventral striatal activation during reward anticipation in adolescent smokers. Am J Psychiatry. 2011; 168:540-549), gambling (e.g. Balodis I M, Kober H, Worhunsky P D, Stevens M C, Pearlson G D, Potenza M N. Diminished fronto-striatal activity during processing of monetary rewards and losses in pathological gambling. Biol Psychiatry. 2012; 71:749-757; Choi J-S, Shin Y-C, Jung W H, Jang J H, Kang D-H, Choi C-H, Choi S-W, Lee J-Y, Hwang J Y, Kwon J S. Altered Brain Activity during Reward Anticipation in Pathological Gambling and Obsessive-Compulsive Disorder. PLoS One. 2012; 7:e45938)] show relatively blunted activation of the ventral striatum, a region implicated in the anticipatory phase of reward processing [e.g. Knutson B, Fong G W, Adams C M, Varner J L, Hommer D. Dissociation of reward anticipation and outcome with event-related fMRI. Neuroreport. 2001; 12:3683-3687; Knutson B, Fong G W, Bennett S M, Adams C M, Hommer D. A region of mesial prefrontal cortex tracks monetarily rewarding outcomes: characterization with rapid event-related fMRI. Neuroimage. 2003; 18:263-272; Knutson B, Adams C M, Fong G W, Hommer D. Anticipation of increasing monetary reward selectively recruits nucleus accumbens. J Neurosci. 2001; 21:RC159 (151-155)]. Further, this blunted activation has been observed in individuals at elevated risk for addictions (those family history positive for alcoholism (e.g. Andrews M M, Meda S A, Thomas A D, Potenza M N, Krystal J H, Worhunsky P, Stevens M C, O'Malley S S, Book G A, Pearlson G D. Individuals Family History Positive for Alcoholism Show fMRI Abnormalities in Reward Sensitivity that are Related to Impulsivity Factors. Biol Psychiatry. 2011; 69:675-683), has been linked to impulsivity (e.g. Beck A, Schlagenhauf F, Wustenberg T, Hein J, Kienast T, Kahnt T, Schmack K, Hagele C, Knutson B, Heinz A, Wrase J. Ventral striatal activation during reward anticipation correlates with impulsivity in alcoholics. Biol Psychiatry. 2009; 66:734-742; Balodis I M, Kober H, Worhunsky P D, Stevens M C, Pearlson G D, Potenza M N. Diminished fronto-striatal activity during processing of monetary rewards and losses in pathological gambling. Biol Psychiatry. 2012; 71:749-757), and may change with treatment (increasing following treatment as compared to before {e.g. Garrison K A, Yip S W, Balodis I M, Carroll K M, Potenza M N, Krishnan-Sarin S. Reward-related frontostriatal activity and smoking behavior among adolescents in treatment for smoking cessation. Drug Alcohol Depend. 2017; 177:268-276}). Similar findings of blunted ventral striatal activation during reward processing have recently been reported in people with internet gaming disorder (Dong G, Li H, Wang L, Potenza M N. Cognitive Control and Reward/Loss Processing in Internet Gaming Disorder: Results from a Comparison with Recreational Internet Game-Users. Eur Psychiatry. 2017; 44:30-38).

### SUMMARY OF THE INVENTION

In one aspect, the invention relates to the use of mavoglurant, or a pharmaceutically acceptable salt thereof:
in the treatment of gambling disorder;
in the treatment of the symptoms of depression or anxiety associated with gambling disorder;
in a treatment in the reduction of gambling by a gambling disorder patient;
in a treatment to prevent relapse into gambling by a gambling disorder patient;
in a treatment to promote gambling abstinence by a gambling disorder patient

In another aspect, the invention relates to the use of mavoglurant, or a pharmaceutically acceptable salt thereof:
in the treatment of gaming disorder;
in the treatment of the symptoms of depression or anxiety associated with gaming disorder;
in a treatment in the reduction of gaming by a gaming disorder patient;
in a treatment to prevent relapse into gaming by a gaming disorder patient;
in a treatment to promote gaming abstinence by a gaming disorder patient

### BRIEF DESCRIPTION OF DRAWINGS

FIG. 1: Schematic diagram depicting our version of the MIDT that includes prospect (A1), anticipation (A2) and outcome (OC) phases for wins and losses that can be appropriately modeled given jitter lengths and placements.

FIG. 2A: Plots from A1 phase of the MIDT, response to losses, in left (L) nucleus accumbens (NAcc)/ventral striatum (VS). Key: dashed lines FHP, solid lines FHN. Drug=mavoglurant.

FIG. 2B: Plots from A1 phase of the MIDT, response to losses, in right (R) nucleus accumbens (NAcc)/ventral striatum (VS). Key: dashed lines FHP, solid lines FHN. Drug=mavoglurant.

### DETAILED DESCRIPTION OF THE INVENTION

In one aspect, mavoglurant may be an ideal candidate for treating patients diagnosed with gambling disorder, having therapeutic advantages for said patient population, such as one or more of the following:
i) promoting gambling abstinence, for example, compared to placebo, for example by maintaining abstinence or by reducing the amount or frequency of gambling, for example as assessed by using self-reported tools, such as the Gambling Timeline Followback (e.g. in Weinstock, J., Whelan, J. P., & Meyers, A. W. (2004). Behavioral Assessment of Gambling: An Application of the Timeline Followback Method. Psychological Assessment, 16(1), 72-80. https://doi.org/10.1037/1040-3590.16.1.72);
ii) decreasing relapse into gambling, for example, compared to placebo, for example it increases the time to relapse or the rates of patient relapse in a treatment program, such as a clinical trial;
iii) alleviating (e.g. by eliminating or by reducing intensity, duration or frequency), for example compared to placebo, one or more of symptoms associated with gambling disorder, such as:
   a. depressive symptoms, for example as assessed from the Beck's Depression Inventory [Beck, A. T. et al., (1961) An inventory for measuring depression. Archives of General Psychiatry, 4, 561-571; Beck, A. T. et al., (1988) Psychometric properties of the Beck Depression Inventory: Twenty-five years of evaluation. Clinical Psychology Review, 8(1), 77-100]; or
   b. anxiety symptoms, for example as assessed from the State-Trait Anxiety Inventory [Spielberger, C. D. (1989). State-Trait Anxiety Inventory: Bibliography (2^{nd} Ed.). Palo Alto, CA: Consulting Psychologists Press; Spielberger, C. D. et al., (1983). Manual for the State-Trait Anxiety Inventory. Palo Alto, CA: Consulting Psychologists Press];
iv) increasing retention of patients in treatment, for example, compared to placebo, for example it increases the rates of patient retention in a treatment program, such as a clinical trial (e.g. as measured by patient attendance at scheduled clinic visits and/or time to dropout from clinical protocol);
v) it improves quality of life, for example compared to placebo, for example as assessed with standard tools; or
vi) it has a favorable therapeutic profile, such as a favorable safety profile or metabolic profile, for example a favorable profile in relation to psychiatric adverse events, genotoxicity, or cardiovascular adverse events (e.g. blood pressure, heart rate, electrocardiography parameters); for example, it has better therapeutic profile (e.g. fewer side effects, decreased off-target effects or decreased toxicity, such as decreased genotoxicity) compared to known therapeutic agent/s that have been tested in the treatment of gambling disorder.

In another aspect, mavoglurant may be an ideal candidate for treating patients diagnosed with gaming disorder, having therapeutic advantages for said patient population, such as one or more of the following:
i) promoting gaming abstinence, for example, compared to placebo, for example by maintaining abstinence or by reducing the amount or frequency of gaming, for example as assessed by using self-reported tools, such as the Assessment of Internet and Computer Game Addiction Self-report (AICA-S; e.g. in JAMA Psychiatry, 2019, 76(10), 1018-1025);
ii) decreasing relapse into gaming, for example, compared to placebo, for example it increases the time to relapse or the rates of patient relapse in a treatment program, such as a clinical trial;
iii) alleviating (e.g. by eliminating or by reducing intensity, duration or frequency), for example compared to placebo, one or more of symptoms associated with gaming disorder, such as:
   a. depressive symptoms, for example as assessed from the Beck's Depression Inventory [Beck, A. T. et al., (1961) An inventory for measuring depression. Archives of General Psychiatry, 4, 561-571; Beck, A. T. et al., (1988) Psychometric properties of the Beck Depression Inventory: Twenty-five years of evaluation. Clinical Psychology Review, 8(1), 77-100]; or
   b. anxiety symptoms, for example as assessed from the State-Trait Anxiety Inventory [Spielberger, C. D. (1989). State-Trait Anxiety Inventory: Bibliography (2^{nd} Ed.). Palo Alto, CA: Consulting Psychologists Press; Spielberger, C. D. et al., (1983). Manual for the State-Trait Anxiety Inventory. Palo Alto, CA: Consulting Psychologists Press];
iv) increasing retention of patients in treatment, for example, compared to placebo, for example it increases the rates of patient retention in a treatment program, such as a clinical trial (e.g. as measured by patient attendance at scheduled clinic visits and/or time to dropout from clinical protocol);
v) it improves quality of life, for example compared to placebo, for example as assessed with standard tools; or
vi) it has a favorable therapeutic profile, such as a favorable safety profile or metabolic profile, for example a favorable profile in relation to psychiatric adverse events, genotoxicity, or cardiovascular adverse events (e.g. blood pressure, heart rate, electrocardiography parameters); for example, it has better therapeutic profile (e.g. fewer side effects, decreased off-target effects or decreased toxicity, such as decreased genotoxicity) compared to known therapeutic agent/s that have been tested in the treatment of gaming disorder.

### Embodiments of the Present Invention are

### Embodiments (a)

1a. Mavoglurant, or a pharmaceutically acceptable salt thereof, for use in the treatment of gambling disorder.
2a. Mavoglurant, or a pharmaceutically acceptable salt thereof, for use in the treatment of the symptoms of depression or anxiety associated with gambling disorder.
3a. Mavoglurant, or a pharmaceutically acceptable salt thereof, for use in the reduction of gambling by a gambling disorder patient.
4a. Use of mavoglurant, or a pharmaceutically acceptable salt thereof, for use in a treatment to prevent relapse into gambling by a gambling disorder patient.
5a. Use of mavoglurant, or a pharmaceutically acceptable salt thereof, for use in a treatment to promote gambling abstinence by a gambling disorder patient.
6a. Mavoglurant, or a pharmaceutically acceptable salt thereof, for use according to any one of embodiments 1a to 5a, wherein gambling disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder, in particular depression or anxiety.
7a. Mavoglurant, or a pharmaceutically acceptable salt thereof, for use according to any one of embodiments 1a to 6a, wherein the gambling disorder is comorbid with substance use disorder (e.g. cocaine use disorder, alcohol use disorder, opioid use disorder or amphetamine-type stimulant use disorder).
8a. Mavoglurant, or a pharmaceutically acceptable salt thereof, for use according to any one of embodiments 1a to 7a, wherein the use is combined with standardized psychological treatment, for example, at individual or group level.
9a. Mavoglurant, or a pharmaceutically acceptable salt thereof, for use according to any one of embodiments 1a to 7a, wherein the use is combined with psychosocial or behavioral therapy or combination thereof, in particular contingency management based therapy.
10a. Mavoglurant, or a pharmaceutically acceptable salt thereof, for use according to embodiment 9a, wherein the psychosocial or the behavioral therapy is computer-assisted.
11a. Mavoglurant, or a pharmaceutically acceptable salt thereof, for use according to any one of embodiments 1a to 10a, wherein mavoglurant, or a pharmaceutically acceptable salt thereof, is administered in combination with a further active agent, for example wherein the further active agent is an antidepressant or an anxiolytic.
12a. Mavoglurant, or a pharmaceutically acceptable salt thereof, for use according to any one of embodiments 1a to 11a, wherein the patient has a genetic variation associated with a substance use disorder.
13a. Mavoglurant, or a pharmaceutically acceptable salt thereof, for use according to any one of embodiments 1a to 12a, wherein mavoglurant, or a pharmaceutically acceptable salt thereof, is administered in an immediate-release form or a modified-release form.
14a. Mavoglurant, or a pharmaceutically acceptable salt thereof, for use according to any one of embodiments 1a to 13a, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15a. Mavoglurant, or a pharmaceutically acceptable salt thereof, for use according to any one of embodiments 1a to 14a, wherein gambling disorder is associated with binge drinking or alcohol use disorder.
16a. Mavoglurant, or a pharmaceutically acceptable salt thereof, for use according to any one of embodiments 1a to 15a, wherein the gambling disorder is mild gambling disorder, moderate gambling disorder or severe gambling disorder.
17a. Mavoglurant, or a pharmaceutically acceptable salt thereof, for use according to any one of embodiments 1a to 15a, wherein the gambling disorder is episodic or persistent.
18a. Mavoglurant, or a pharmaceutically acceptable salt thereof, for use according to any one of embodiments 1a to 15a, wherein the gambling disorder is in early remission or in sustained remission.

### Embodiments (b)

1 b. A method for treating gambling disorder, in a patient in need thereof, comprising administering to said patient mavoglurant, or a pharmaceutically acceptable salt thereof.
2b. A method for treating the symptoms of depression or anxiety associated with gambling disorder, in a patient in need thereof, comprising administering to said patient mavoglurant, or a pharmaceutically acceptable salt thereof.
3b. A method for the reduction of gambling by a gambling disorder patient, in need thereof, comprising administering to said patient mavoglurant, or a pharmaceutically acceptable salt thereof.
4b. A method for preventing relapse into gambling by a gambling disorder patient, in need thereof, comprising administering to said patient mavoglurant, or a pharmaceutically acceptable salt thereof.
5b. A method for the promotion of gambling abstinence by a gambling disorder patient, in need thereof, comprising administering to said patient mavoglurant, or a pharmaceutically acceptable salt thereof.
6b. The method according to any one of embodiments 1b to 5b, wherein gambling disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder, in particular depression or anxiety.
7b. The method according to any one of embodiments 1b to 6b, wherein the gambling disorder is comorbid with substance use disorder (e.g. cocaine use disorder, alcohol use disorder, opioid use disorder or amphetamine-type stimulant use disorder).
8b. The method according to any one of embodiments 1b to 7b, wherein the use is combined with standardized psychological treatment, for example, at individual or group level.
9b. The method according to any one of embodiments 1b to 7b, wherein the use is combined with psychosocial or behavioral therapy or combination thereof, in particular contingency management based therapy.
10b. The method according to embodiment 9b, wherein the psychosocial or the behavioral therapy is computer-assisted.
11b. The method according to any one of embodiments 1b to 10b, wherein mavoglurant, or a pharmaceutically acceptable salt thereof, is administered in combination with a further active agent, for example wherein the further active agent is an antidepressant or an anxiolytic.
12b. The method according to any one of embodiments 1b to 11b, wherein the patient has a genetic variation associated with a substance use disorder.
13b. The method according to any one of embodiments 1b to 12b, wherein mavoglurant, or a pharmaceutically acceptable salt thereof, is administered in an immediate-release form or a modified-release form.
14b. The method according to any one of embodiments 1b to 13b, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15b. The method according to any one of embodiments 1b to 14b, wherein gambling disorder is associated with binge drinking or alcohol use disorder.
16b. The method according to any one of embodiments 1b to 15b, wherein the gambling disorder is mild gambling disorder, moderate gambling disorder or severe gambling disorder.
17b. The method according to any one of embodiments 1b to 15b, wherein the gambling disorder is episodic or persistent.
18b. The method according to any one of embodiments 1b to 15b, wherein the gambling disorder is in early remission or in sustained remission.

### Embodiments (c)

1c. Use of mavoglurant, or a pharmaceutically acceptable salt thereof, for the manufacture of a medicament for the treatment of gaming disorder.
2c. Use of mavoglurant, or a pharmaceutically acceptable salt thereof, for the manufacture of a medicament for the treatment of the symptoms of depression or anxiety associated with gaming disorder.
3c. Use of mavoglurant, or a pharmaceutically acceptable salt thereof, for the manufacture of a medicament in a treatment for the reduction of gaming by a gaming disorder patient.
4c. Use of mavoglurant, or a pharmaceutically acceptable salt thereof, for the manufacture of a medicament in a treatment to prevent relapse into gaming by a gaming disorder patient.
5c. Use of mavoglurant, or a pharmaceutically acceptable salt thereof, for the manufacture of a medicament in a treatment to promote gaming abstinence by a gaming disorder patient.
6c. Use of mavoglurant, or a pharmaceutically acceptable salt thereof, according to any one of embodiments 1c to 5c, wherein gaming disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder, in particular depression or anxiety.
7c. Use of mavoglurant, or a pharmaceutically acceptable salt thereof, according to any one of embodiments 1c to 6c, wherein the gaming disorder is comorbid with substance use disorder (e.g. cocaine use disorder, alcohol use disorder, opioid use disorder or amphetamine-type stimulant use disorder).
8c. Use of mavoglurant, or a pharmaceutically acceptable salt thereof, according to any one of embodiments 1c to 7c, wherein the use is combined with standardized psychological treatment, for example, at individual or group level.
9c. Use of mavoglurant, or a pharmaceutically acceptable salt thereof, according to any one of embodiments 1c to 7c, wherein the use is combined with psychosocial or behavioral therapy or combination thereof, in particular contingency management based therapy.
10c. Use of mavoglurant, or a pharmaceutically acceptable salt thereof, according to embodiment 9c, wherein the psychosocial or the behavioral therapy is computer-assisted.
11c. Use of mavoglurant, or a pharmaceutically acceptable salt thereof, according to any one of embodiments 1c to 10c, wherein mavoglurant, or a pharmaceutically acceptable salt thereof, is administered in combination with a further active agent, for example wherein the further active agent is an antidepressant or an anxiolytic.
12c. Use of mavoglurant, or a pharmaceutically acceptable salt thereof, according to any one of embodiments 1c to 11c, wherein the patient has a genetic variation associated with a substance use disorder.
13c. Use of mavoglurant, or a pharmaceutically acceptable salt thereof, according to any one of embodiments 1c to 12c, wherein mavoglurant, or a pharmaceutically acceptable salt thereof, is administered in an immediate-release form or a modified-release form.
14c. Use of mavoglurant, or a pharmaceutically acceptable salt thereof, according to any one of embodiments 1c to 13c, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15c. Use of mavoglurant, or a pharmaceutically acceptable salt thereof, according to any one of embodiments 1c to 14c, wherein gaming disorder is associated with binge drinking or alcohol use disorder.
16c. Use of mavoglurant, or a pharmaceutically acceptable salt thereof, according to any one of embodiments 1c to 15c, wherein the gaming disorder is mild gaming disorder, moderate gaming disorder or severe gaming disorder.
17c. Use of mavoglurant, or a pharmaceutically acceptable salt thereof, according to any one of embodiments 1c to 15c, wherein the gaming disorder is episodic or persistent.
18c. Use of mavoglurant, or a pharmaceutically acceptable salt thereof, according to any one of embodiments 1c to 15c, wherein the gaming disorder is in early remission or in sustained remission.

### Embodiments (d)

1d. Mavoglurant, or a pharmaceutically acceptable salt thereof, for use in the treatment of gaming disorder.
2d. Mavoglurant, or a pharmaceutically acceptable salt thereof, for use in the treatment of the symptoms of depression or anxiety associated with gaming disorder.
3d. Mavoglurant, or a pharmaceutically acceptable salt thereof, for use in the reduction of gaming by a gaming disorder patient.
4d. Use of mavoglurant, or a pharmaceutically acceptable salt thereof, for use in a treatment to prevent relapse into gaming by a gaming disorder patient.
5d. Use of mavoglurant, or a pharmaceutically acceptable salt thereof, for use in a treatment to promote gaming abstinence by a gaming disorder patient.
6d. Mavoglurant, or a pharmaceutically acceptable salt thereof, for use according to any one of embodiments 1d to 5d, wherein gaming disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder, in particular depression or anxiety.
7d. Mavoglurant, or a pharmaceutically acceptable salt thereof, for use according to any one of embodiments 1d to 6d, wherein the gaming disorder is comorbid with substance use disorder (e.g. cocaine use disorder, alcohol use disorder, opioid use disorder or amphetamine-type stimulant use disorder).
8d. Mavoglurant, or a pharmaceutically acceptable salt thereof, for use according to any one of embodiments 1d to 7d, wherein the use is combined with standardized psychological treatment, for example, at individual or group level.
9d. Mavoglurant, or a pharmaceutically acceptable salt thereof, for use according to any one of embodiments 1d to 7d, wherein the use is combined with psychosocial or behavioral therapy or combination thereof, in particular contingency management based therapy.
10d. Mavoglurant, or a pharmaceutically acceptable salt thereof, for use according to embodiment 9d, wherein the psychosocial or the behavioral therapy is computer-assisted.
11d. Mavoglurant, or a pharmaceutically acceptable salt thereof, for use according to any one of embodiments 1d to 10d, wherein mavoglurant, or a pharmaceutically acceptable salt thereof, is administered in combination with a further active agent, for example wherein the further active agent is an antidepressant or an anxiolytic.
12d. Mavoglurant, or a pharmaceutically acceptable salt thereof, for use according to any one of embodiments 1d to 11d, wherein the patient has a genetic variation associated with a substance use disorder.
13d. Mavoglurant, or a pharmaceutically acceptable salt thereof, for use according to any one of embodiments 1d to 12d, wherein mavoglurant, or a pharmaceutically acceptable salt thereof, is administered in an immediate-release form or a modified-release form.
14d. Mavoglurant, or a pharmaceutically acceptable salt thereof, for use according to any one of embodiments 1d to 13d, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15d. Mavoglurant, or a pharmaceutically acceptable salt thereof, for use according to any one of embodiments 1d to 14d, wherein gaming disorder is associated with binge drinking or alcohol use disorder.
16d. Mavoglurant, or a pharmaceutically acceptable salt thereof, for use according to any one of embodiments 1d to 15d, wherein the gaming disorder is mild gaming disorder, moderate gaming disorder or severe gaming disorder.
17d. Mavoglurant, or a pharmaceutically acceptable salt thereof, for use according to any one of embodiments 1d to 15d, wherein the gaming disorder is episodic or persistent.
18d. Mavoglurant, or a pharmaceutically acceptable salt thereof, for use according to any one of embodiments 1d to 15d, wherein the gaming disorder is in early remission or in sustained remission.

### Embodiments (e)

1e. A method for treating gaming disorder, in a patient in need thereof, comprising administering to said patient mavoglurant, or a pharmaceutically acceptable salt thereof.
2e. A method for treating the symptoms of depression or anxiety associated with gaming disorder, in a patient in need thereof, comprising administering to said patient mavoglurant, or a pharmaceutically acceptable salt thereof.
3e. A method for the reduction of gaming by a gaming disorder patient, in need thereof, comprising administering to said patient mavoglurant, or a pharmaceutically acceptable salt thereof.
4e. A method for preventing relapse into gaming by a gaming disorder patient, in need thereof, comprising administering to said patient mavoglurant, or a pharmaceutically acceptable salt thereof.
5e. A method for the promotion of gaming abstinence by a gaming disorder patient, in need thereof, comprising administering to said patient mavoglurant, or a pharmaceutically acceptable salt thereof.
6e. The method according to any one of embodiments 1e to 5e, wherein gaming disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder, in particular depression or anxiety.
7e. The method according to any one of embodiments le to 6e, wherein the gaming disorder is comorbid with substance use disorder (e.g. cocaine use disorder, alcohol use disorder, opioid use disorder or amphetamine-type stimulant use disorder).
8e. The method according to any one of embodiments 1e to 7e, wherein the use is combined with standardized psychological treatment, for example, at individual or group level.
9e. The method according to any one of embodiments 1e to 7e, wherein the use is combined with psychosocial or behavioral therapy or combination thereof, in particular contingency management based therapy.
10e. The method according to embodiment 9e, wherein the psychosocial or the behavioral therapy is computer-assisted.
11e. The method according to any one of embodiments 1e to 10e, wherein mavoglurant, or a pharmaceutically acceptable salt thereof, is administered in combination with a further active agent, for example wherein the further active agent is an antidepressant or an anxiolytic.
12e. The method according to any one of embodiments 1e to 11e, wherein the patient has a genetic variation associated with a substance use disorder.
13e. The method according to any one of embodiments 1e to 12e, wherein mavoglurant, or a pharmaceutically acceptable salt thereof, is administered in an immediate-release form or a modified-release form.
14e. The method according to any one of embodiments 1e to 13e, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.
15e. The method according to any one of embodiments 1e to 14e, wherein gaming disorder is associated with binge drinking or alcohol use disorder.
16e. The method according to any one of embodiments 1e to 15e, wherein the gaming disorder is mild gaming disorder, moderate gaming disorder or severe gaming disorder.
17e. The method according to any one of embodiments 1e to 15e, wherein the gaming disorder is episodic or persistent.
18e. The method according to any one of embodiments 1e to 15e, wherein the gaming disorder is in early remission or in sustained remission.

### GENERAL TERMS

The term “gambling disorder”, as used herein, refers to, for example, the definition provided with reference to diagnostic criteria such as DSM-5 criteria (i.e. according to the Diagnostic and Statistical Manual of Mental Disorders. 5^{th} Edition, Washington, DC: American Psychiatric Association, 2013), the entire contents of which, in particular contents of the section on “gambling disorder” are incorporated herein by reference, in particular as follows:

A. Persistent and recurrent problematic gambling behavior leading to clinically significant impairment or distress, as indicated by the individual exhibiting four (or more) of the following in a 12-month period:
1. Needs to gamble with increasing amounts of money in order to achieve the desired excitement.
2. Is restless or irritable when attempting to cut down or stop gambling.
3. Has made repeated unsuccessful efforts to control, cut back, or stop gambling.
4. Is often preoccupied with gambling (e.g., having persistent thoughts of reliving past gambling experiences, handicapping or planning the next venture, thinking of ways to get money with which to gamble).
5. Often gambles when feeling distressed (e.g., helpless, guilty, anxious, depressed).
6. After losing money gambling, often returns another day to get even (“chasing” one's losses).
7. Lies to conceal the extent of involvement with gambling.
8. Has jeopardized or lost a significant relationship, job, or educational or career opportunity because of gambling.
9. Relies on others to provide money to relieve desperate financial situations caused by gambling.
   B: The gambling behavior is not better explained by a manic episode
   “Gambling disorder” may be separated into the following three categories: mild (e.g. presence of 4 to 5 symptoms, defined with reference to DSM-5 criteria), moderate (e.g. presence of 6 to 7 symptoms, defined with reference to DSM-5 criteria) and severe (e.g. presence of 8 or 9 symptoms, defined with reference to DSM-5 criteria). In one embodiment “gambling disorder”, as used herein, refers to “mild gambling disorder”, “moderate gambling disorder” and “severe gambling disorder.

The term “gambling”, as used herein, refers, for example, to risking something of value in the hopes of obtaining something of greater value. As per definition of the DSM-5, included herein above, for gambling disorder diagnosis (Criterion A) the gambling behaviour must be a persistent and recurrent maladaptive gambling behavior that disrupts personal, family, and/or vocational pursuits. Gambling disorder is defined as a cluster of four or more of the symptoms listed in Criterion A occurring at any time in the same 12-month period.

The term “gambling disorder patient”, as used herein, refers to a patient diagnosed with gambling disorder, for example, as defined herein.

In one embodiment, the term “gambling disorder patient” refers to a patient diagnosed with gambling disorder, who is in abstinence from gambling, for example, for at least 1 day, such as 3 days or more. The term “gambling disorder patient in abstinence” refers to a patient diagnosed with gambling disorder, for example, as defined herein, in abstinence from gambling for a period, for example, for at least 1 day.

The term “gambling disorder associated with binge drinking” refers to a patient who is diagnosed with gambling disorder, for example, as defined herein, and is an abuser of alcohol (i.e. a heavy drinker). As explained at http://drugabuse.com/library/alcohol-abuse/, abusers of alcohol may not drink on a consistent basis, for example, they may only drink once a week, but, when drinking, they may drink heavily, which will cause problems, such as suffering from alcohol intoxication. For the sake of clarity, herein, an abuser of alcohol is not an alcohol use disorder patient (i.e. does not meet criteria for alcohol use disorder as defined with reference to DSM-5 criteria). The term “heavy drinker” refers to someone with a heavy alcohol use pattern. According to the National Institute on Alcohol Abuse and Alcoholism (NIAAA), the Substance Abuse and Mental Health Services Administration (SAMHSA) defines “heavy alcohol use” as binge drinking on 5 or more days in the past month. NIAAA defines binge drinking as a pattern of drinking that brings blood alcohol concentration (BAC) levels to 0.08 g/dL. This typically occurs after 4 alcoholic drinks for women and 5 alcoholic drinks for men—in about 2 hours. The Substance Abuse and Mental Health Services Administration (SAMHSA), defines “binge drinking” as 5 or more alcoholic drinks for males or 4 or more alcoholic drinks for females on the same occasion (i.e., at the same time or within a couple of hours of each other) on at least 1 day in the past month. The term “alcohol”, as used herein, for example in relation to “drinks”, “alcoholic drinks” or “drinking”, refers to ethyl alcohol (i.e. ethanol). The term “drinking”, “drinks” or “alcoholic drinks”, as used herein, is understood in the context of “standard drinks”, such as spirits or blends that are intended for human consumption, wherein a “standard drink” equals 12 g ethanol.

The term “gambling disorder associated with substance use disorder (e.g. alcohol use disorder)” refers to a patient who is diagnosed with gambling disorder, for example, as defined herein, and who is also diagnosed with substance use disorder (e.g. alcohol use disorder) [i.e. it meets criteria for substance use disorder (e.g. alcohol use disorder), for example as defined with reference to DSM-5 criteria i.e. according to the Diagnostic and Statistical Manual of Mental Disorders. 5^{th} Edition, Washington, DC: American Psychiatric Association, 2013, the entire contents of which, in particular contents of the section on “substance use disorder” (e.g. alcohol use disorder), are incorporated herein by reference].

The term “reducing gambling” or “reduction of gambling”, as used herein, refers, for example, to reducing the time amount or frequency of gambling, wherein gambling is, for example. as defined herein, for example, as assessed by using a self-reported tool, such as standardized tools such as the Gambling Timeline Followback [e.g. in Weinstock, J., Whelan, J. P., & Meyers, A. W. (2004). Behavioral Assessment of Gambling: An Application of the Timeline Followback Method. Psychological Assessment, 16(1), 72-80. https://doi.org/10.1037/1040-3590.16.1.72].

In one embodiment, “reducing gambling” or “reduction of gambling”, as used herein, refers to “reducing gambling craving”, for example, by assessment of the reduction in the level of urge to gamble with an standardized tool, such as the Yale Brown Obsessive-Compulsive Scale Modified for Pathological Gambling (PG-YBOCS) [e.g. in Pallanti S, DeCaria C M, Grant J E, Urpe M, Hollander E. Reliability and validity of the pathological gambling adaptation of the Yale-Brown Obsessive-Compulsive Scale (PG-YBOCS). J Gambling Stud. 2005; 21:431-443].

The term “gambling abstinence” or “in abstinence from gambling”, as used herein, refers, for example, to not gambling. The term “promoting gambling abstinence” or “promotion of gambling abstinence”, as used herein, refers for example, to help maintaining abstinence from gambling. in particular after at least 1 day of not gambling, for example maintaining abstinence from gambling for a period of, for example, at least 1 week, 2 weeks, 3 weeks, 1 month, 3 months, 6 months or more, in particular at least 1 week or more, such as 2 weeks.

The term “relapse into gambling”, as used herein, refers, for example, to gambling following a period of gambling abstinence, for example following a period of gambling abstinence of at least 1 day or more, such as 3 days, 1 week, 2 weeks, 3 weeks, 1 month, 3 months, 6 months or more.

The term “preventing relapse into gambling”, as used herein, refers, for example, to the prevention of gambling by a gambling disorder patient, for example, as defined herein, after the patient has stopped gambling, in particular after 1 day or more of not gambling. In some embodiments, the term encompasses the permanent stoppage of gambling. In other embodiments, the term encompasses a delay in the resumption of gambling as compared to the time to resumption by a subject that is not administered a compound of the invention. The delay in resumption can be, e.g., days (e.g., 2, 3, 4, 5, 6, 7 days), weeks (e.g., 1, 2, 3 weeks), months (e.g., 1, 2, 3, 4, 5, 6 months), or longer.

The expression “gambling disorder is episodic”, as used herein, refers to, for example, meeting diagnostic criteria at more than one time point, with symptoms subsiding between periods of gambling disorder for at least several months.

The expression “gambling disorder is persistent”, as used herein, refers to, for example, experiencing continuous symptoms, to meet diagnostic criteria for multiple years.

The expression “gambling disorder is in sustained remission”, as used herein, means, for example, that after full criteria for gambling disorder were previously met, none of the criteria for gambling disorder have been met during a period of 12 months or longer.

The expression “gambling disorder is in early remission”, as used herein, means, for example, that after full criteria for gambling disorder were previously met, none of the criteria for gambling disorder have been met for at least 3 months but for less than 12 months.

The term “gaming disorder”, as used herein, refers, for example, to the definition provided in the ICD-11 (i.e. International Classification of Disease, 11^{th} Revision; e.g in Version 9/2020 available at https://icd.who.int/browse11/l-m/en#/http://id.who.int/icd/entity/1448597234), the entire contents of which, in particular contents of the section on 6C51on “Gaming disorder”, are incorporated herein by reference, in particular as follows:

Gaming disorder is characterised by a pattern of persistent or recurrent gaming behaviour (‘digital gaming’ or ‘video-gaming’), which may be online (i.e., over the internet) or offline, manifested by: 1. impaired control over gaming (e.g., onset, frequency, intensity, duration, termination, context); 2. increasing priority given to gaming to the extent that gaming takes precedence over other life interests and daily activities; and 3. continuation or escalation of gaming despite the occurrence of negative consequences. The pattern of gaming behaviour may be continuous or episodic and recurrent. The pattern of gaming behaviour results in marked distress or significant impairment in personal, family, social, educational, occupational, or other important areas of functioning. The gaming behaviour and other features are normally evident over a period of at least 12 months in order for a diagnosis to be assigned, although the required duration may be shortened if all diagnostic requirements are met and symptoms are severe.
In one embodiment, gaming disorder, is to be understood in accordance to research criteria for internet gaming disorder as defined in Section III in DSM-5, which are incorporated herein by reference.
“Gaming disorder” may be separated into the following three categories: mild, moderate and severe. In one embodiment “gaming disorder”, as used herein, refers to “mild gaming disorder”, “moderate gaming disorder” and “severe gaming disorder.

The term “gaming”, as used herein, refers, for example, to ‘digital gaming’ or ‘video gaming’. As per definition of the ICD-11, included herein above, for gaming disorder diagnosis .

The term “gaming disorder patient”, as used herein, refers to a patient diagnosed with gaming disorder, for example, as defined herein.

In one embodiment, the term “gaming disorder patient” refers to a patient diagnosed with gaming disorder, who is in abstinence from gaming, for example, for at least 1 day, such as 3 days or more. The term “gaming disorder patient in abstinence” refers to a patient diagnosed with gaming disorder, as defined herein, in abstinence from gaming for a period, for example, for at least 1 day. The term “gaming disorder associated with binge drinking” refers to a patient who is diagnosed with gaming disorder, for example, as defined herein, and is an abuser of alcohol (i.e. a heavy drinker). As explained, for example, at http://drugabuse.com/library/alcohol-abuse/, abusers of alcohol may not drink on a consistent basis, for example, they may only drink once a week, but, when drinking, they may drink heavily, which will cause problems, such as suffering from alcohol intoxication. For the sake of clarity, herein, an abuser of alcohol is not an alcohol use disorder patient (i.e. does not meet criteria for alcohol use disorder as defined with reference to DSM-5 criteria). The term “heavy drinker” refers to someone with a heavy alcohol use pattern. According to the National Institute on Alcohol Abuse and Alcoholism (NIAAA), the Substance Abuse and Mental Health Services Administration (SAMHSA) defines “heavy alcohol use” as binge drinking on 5 or more days in the past month. NIAAA defines binge drinking as a pattern of drinking that brings blood alcohol concentration (BAC) levels to 0.08 g/dL. This typically occurs after 4 alcoholic drinks for women and 5 alcoholic drinks for men—in about 2 hours. The Substance Abuse and Mental Health Services Administration (SAMHSA), defines “binge drinking” as 5 or more alcoholic drinks for males or 4 or more alcoholic drinks for females on the same occasion (i.e., at the same time or within a couple of hours of each other) on at least 1 day in the past month. The term “alcohol”, as used herein, for example in relation to “drinks”, “alcoholic drinks” or “drinking”, refers to ethyl alcohol (i.e. ethanol). The term “drinking”, “drinks” or “alcoholic drinks”, as used herein, is understood in the context of “standard drinks”, such as spirits or blends that are intended for human consumption, wherein a “standard drink” equals 12 g ethanol.

The term “gaming disorder associated with substance use disorder” (e.g. alcohol use disorder) refers to a patient who is diagnosed with gaming disorder, for example, as defined herein, and who is also diagnosed with substance use disorder (e.g. alcohol use disorder) [i.e. it meets criteria for substance use disorder (e.g. alcohol use disorder), for example as defined with reference to DSM-5 criteria i.e. according to the Diagnostic and Statistical Manual of Mental Disorders. 5^{th} Edition, Washington, DC: American Psychiatric Association, 2013, the entire contents of which, in particular contents of the section on “substance use disorder” (e.g. alcohol use disorder), are incorporated herein by reference].

The term “reducing gaming” or “reduction of gaming”, as used herein, refers, for example, to reducing the time amount or frequency of gaming, wherein gaming is for example, as defined herein, for example as assessed by using a self-reported tool.

In one embodiment, “reducing gaming” or “reduction of gaming”, as used herein, refers to “reducing gaming craving”, for example, by assessment of the reduction in the level of urge to game with an standardized tool, such as the Assessment of Internet and Computer Game Addiction Self-report (AICA-S; e.g. in JAMA Psychiatry, 2019, 76(10), 1018-1025) or the short clinical interview checklist for the Assessment of Internet and Computer game Addiction (AICA-C; e.g. in J Addict Res Ther S6:003 doi:10.4172/2155-6105.S6-003).

The term “gaming abstinence” or “in abstinence from gaming”, as used herein, refers, for example, to not gaming, as defined herein. The term “promoting gaming abstinence” or “promotion of gaming abstinence”, as used herein, refers, for example, to help maintaining abstinence from gaming, as defined herein, in particular after at least 1 day of not gaming, as defined herein, for example maintaining abstinence from gaming for a period of, for example, at least 1 week, 2 weeks, 3 weeks, 1 month, 3 months, 6 months or more, in particular at least 1 week or more, such as 2 weeks.

The term “relapse into gaming”, as used herein, refers, for example, to gaming following a period of gaming abstinence, for example following a period of gaming abstinence of at least 1 day or more, such as 3 days, 1 week, 2 weeks, 3 weeks, 1 month, 3 months, 6 months or more.

The term “preventing relapse into gaming”, as used herein, refers, for example, to the prevention of gaming by a gaming disorder patient, for example, as defined herein, after the patient has stopped gaming, in particular after 1 day or more of not gaming. In some embodiments, the term encompasses the permanent stoppage of gaming. In other embodiments, the term encompasses a delay in the resumption of gaming as compared to the time to resumption by a subject that is not administered a compound of the invention. The delay in resumption can be, e.g., days (e.g., 2, 3, 4, 5, 6, 7 days), weeks (e.g., 1, 2, 3 weeks), months (e.g., 1, 2, 3, 4, 5, 6 months), or longer.

The expression “gaming disorder is episodic”, as used herein, refers to, for example, meeting diagnostic criteria at more than one time point, with symptoms subsiding between periods of gaming disorder for at least several months.

The expression “gaming disorder is persistent”, as used herein, refers to, for example, experiencing continuous symptoms, to meet diagnostic criteria for multiple years.

The expression “gaming disorder is in sustained remission”, as used herein, means, for example, that after full criteria for gaming disorder were previously met, none of the criteria for gaming disorder have been met during a period of 12 months or longer.

The expression “gaming disorder is in early remission”, as used herein, means, for example, that after full criteria for gaming disorder were previously met, none of the criteria for gaming disorder have been met for at least 3 months but for less than 12 months.

The term “antidepressant”, as used herein, refers to an active ingredient commonly used to treat depression, such as a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g. amitriptyline, nortriptyline, doxepin, desipramine, imipramine, protriptyline, trimipramine, clomipramine), a tetracyclic antidepressant (e.g. maprotiline, mianserin, mirtazapine, setiptiline), or a monoamine oxidase inhibitor (MAOI, e.g. isocarboxazid, phenelzine, selegiline, tranylcypromine). In one embodiment, the antidepressant is selected from the group consisting of a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g. amitriptyline, nortriptyline, doxepin, desipramine, imipramine, protriptyline, trimipramine, clomipramine), a tetracyclic antidepressant (e.g. maprotiline, mianserin, mirtazapine, setiptiline), a monoamine oxidase inhibitor (MAOI, e.g. isocarboxazid, phenelzine, selegiline, tranylcypromine) and hypericum perforatum. In another embodiment, the antidepressant is selected from the group consisting of fluoxetine, citalopram, sertraline, paroxetine, escitalopram, fluvoxamine, vilazodone, vortioxetine, venlafaxine, duloxetine, desvenlafaxine, milnacipran, levomilnacipran, bupropion, amitriptyline, nortriptyline, doxepin, desipramine, imipramine, protriptyline, trimipramine, clomipramine, maprotiline, mianserin, mirtazapine, setiptiline, isocarboxazid, phenelzine, selegiline, tranylcypromine and hypericum perforatum; or salts thereof. In another embodiment, the antidepressant is selected from the group consisting of fluoxetine, citalopram, sertraline, paroxetine, escitalopram, fluvoxamine, vilazodone, vortioxetine, venlafaxine, duloxetine, desvenlafaxine, milnacipran, levomilnacipran, bupropion, amitriptyline, nortriptyline, doxepin, desipramine, imipramine, protriptyline, trimipramine, clomipramine, maprotiline, mianserin, mirtazapine, setiptiline, isocarboxazid, phenelzine, selegiline and tranylcypromine; or salts thereof.

The term “anxiolytic”, as used herein, refers to a drug that inhibits anxiety, such as benzodiazepines (e.g. alprazolam, bromazepam, chlordiazepoxide, clonazepam, clorazepate, diazepam, flurazepam, lorazepam, oxazepam, temazepam, triazolam) or antihistamines (e.g. hydroxyzine). In one embodiment, the anxiolytic is selected from the group consisting of alprazolam, bromazepam, chlordiazepoxide, clonazepam, clorazepate, diazepam, flurazepam, lorazepam, oxazepam, temazepam, triazolam, and hydroxyzine; or salts thereof.

The term “psychosocial or behavioral therapy”, as used herein, refers to, but not limited to, cognitive behavioral therapy (e.g. as described in Arch. Gen. Psychiatry 1999; 56:493-502), interpersonal therapy (e.g. as described in Psychol Addict Behav 2009; 23(1): 168-174), contingency management based therapy (e.g. as described in Psychol Addict Behav 2009; 23(1): 168-174; in J. Consul. Clin. Psychol. 2005; 73(2): 354-59; or in Case Reports in Psychiatry, Vol. 2012, Article ID 731638), community reinforcement approach based therapy (e.g. as described in Drug Alcohol Depend 2004; 74:1 -13), motivational interviewing based therapy (e.g. as described in J. Consul. Clin. Psychol. 2001; 69(5): 858-62), motivational enhancement based therapy (e.g. as described in Drug Alcohol Depend 2007, 91:97-101 ) or meditation based therapy, such as transcendental meditation based therapy (e.g. as described in Addiction 2004; 99(7):862-874 or J. Consul. Clin. Psychol. 2000; 68(3): 515-52).

The term “standardized psychological treatment” or ““standardized psychological support”, as used herein, refers to, for example, standard counselling sessions, for example once a week, in one particular aspect, counselling focused on gambling. In another aspect, it refers to, for example, standard counselling sessions, for example once a week, in another particular aspect, counselling focused on gaming.

The term “computer-assisted” in the expression “the psychosocial or the behavioral therapy is computer-assisted”, as used herein, refers to, for example, psychosocial or behavioral therapy comprising the use of electronic tools such as online tools, smartphones, wireless devices or health Apps. In one embodiment, the term “computer-assisted” in the expression “the psychosocial or the behavioral therapy is computer-assisted”, as used herein, is to be understood as “computer-implemented” (i.e. the psychosocial or the behavioral therapy is computer-implemented).

The term “administered with food” refers to, for example, any food product, solid or liquid, with caloric content. The dosage of the mavoglurant, or pharmaceutically acceptable salt thereof, may be administered to a subject, for example, between thirty minutes prior to eating food, to, for example, one hour after consumption. For example, administration of mavoglurant, or pharmaceutically acceptable salt thereof, occurs immediately after consuming food up to about thirty minutes after consumption.

The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations. In one embodiment, the genetic variation is a genetic variation in mGluR5.

The term “treat” “treating” “treatment” or “therapy”, as used herein, means obtaining beneficial or desired results, for example, clinical results. In one aspect, beneficial or desired results can include, but are not limited to, alleviation of one or more symptoms of gambling disorder patients, as defined herein, such as anxiety symptoms or depression symptoms associated with gambling disorder, as defined herein, in particular by a gambling disorder patient, as defined herein, in abstinence from gambling, as herein defined. In another aspect, beneficial or desired results can include, but are not limited to, alleviation of one or more symptoms of gaming disorder patients, as defined herein, such as anxiety symptoms or depression symptoms associated with gaming disorder, as defined herein, in particular by a gaming disorder patient, as defined herein, in abstinence from gaming, as herein defined. One aspect of the treatment is, for example, that said treatment should have a minimal adverse effect on the patient, e.g. the agent used should have a high level of safety, for example without producing adverse side effects. The term “alleviation”, for example in reference to a symptom of a condition, as used herein, refers to, for example, reducing at least one of the frequency and amplitude of a symptom of a condition in a patient.

As used herein, the term “subject” refers to a mammalian organism, preferably a human being (male or female).

As used herein, the term “patient” refers to a subject who is diseased and would benefit from the treatment.

As used herein, a subject is “in need of” a treatment if such a subject (patient) would benefit biologically, medically or in quality of life from such a treatment.

The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.

The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein. In particular, reference to mavoglurant, or a pharmaceutically acceptable salt thereof, in combination with a further active agent, as used herein (e.g. in any of embodiments herein above, or in any of the claims, herein below), refers to mavoglurant in combination with at least one further active agent, for example selected from the group consisting of an antidepressant, an antipsychotic and an anxiolytic.

The term “immediate release form” refers to a pharmaceutical composition designed to release the active substance immediately upon in vivo administration.

The term “modified release form” refers to a pharmaceutical composition which releases the active substance not immediately, but offers a sustained, retard, continuous, gradual, prolonged or pulsatile release and therefore alters drug plasma levels distinctively versus an immediate release form. The term “modified release form” encompasses forms that are described as controlled-release form, sustained-release form, extended-release form, and long-acting form; in particular a sustained-release form.

The term “combination” or “pharmaceutical combination” refers to either a fixed combination in one unit dosage form (e.g., capsule, tablet, caplets or particulates), non-fixed combination, or a kit of parts for the combined administration where a compound of the present invention and one or more combination partner (e.g. another drug as specified herein, also referred to as further “pharmaceutical active ingredient”, “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. the compound of the present invention and one or more combination partners, are both administered to a patient simultaneously in the form of a single entity or dosage. The term “non-fixed combination” means that the active ingredients, e.g. a compound of the present invention and one or more combination partners, are both administered to a patient as separate entities either simultaneously or sequentially with no specific time limits, wherein such administration provides therapeutically effective levels of the two compounds in the body of the patient.

The compound of the present invention may be administered separately, by the same or different route of administration, or together in the same pharmaceutical composition as the other agents. In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

As used herein, the term “a,” “an,” “the” and similar terms used in the context of the present invention (especially in the context of the claims) are to be construed to cover both the singular and plural unless otherwise indicated herein or clearly contradicted by the context.

The use of any and all examples, or exemplary language (e.g. “such as”) provided herein is intended merely to better illuminate the invention and does not pose a limitation on the scope of the invention otherwise claimed.

As used herein, the compound of the invention, alternatively named Compound (I), as used herein above and below, is the mGluR5 antagonist (-)-(3aR,4S,7aR)-4-Hydroxy-4-m-tolylethynyl-octahydro-indole-1-carboxylic acid methyl ester, also named (-)-(3aR,4S,7aR)-4-Hydroxy-4-[2-(3-methylphenyl)ethynyl]perhydroindole-1-carboxylic acid methyl ester, also known as mavoglurant, of formula:

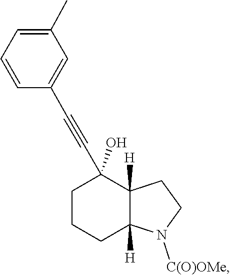

which can be e.g. prepared as described in WO2003/047581, e.g., in Example 1, or as described in WO2010/018154. WO2003/047581, which is incorporated herein by reference, also describes its in-vitro biological data, as per page 7. As used herein, “mavoglurant” refers to the free form, and any reference to “a pharmaceutically acceptable salt thereof” refers to a pharmaceutically acceptable acid addition salt thereof. As used herein, the term “mavoglurant, or a salt thereof, such as a pharmaceutically acceptable salt thereof”, as used in the context of the present invention (especially in the context of the any of the embodiments, above or below, and the claims) is thus to be construed to cover both the free form and a pharmaceutically acceptable salt thereof, unless otherwise indicated herein.

In one embodiment, Compound (I) is also intended to represent isotopically labeled forms. Isotopically labeled compounds have structures depicted by the formula above except that one or more atoms are replaced by an atom having a selected atomic mass or mass number. Isotopes that can be incorporated into the compound of the invention include, for example, isotopes of hydrogen, namely the compound of formula:

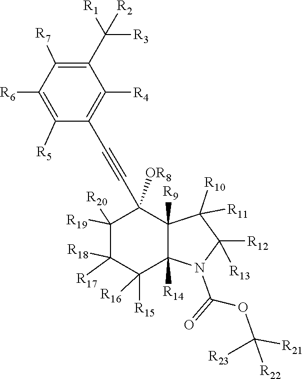

wherein each R₁, R₂, R₃, R₄, R₅, R₆, R₇, R₈, R₉, R₁₀, R₁₁, R₁₂, R₁₃, R₁₄, R₁₅, R₁₆, R₁₇, R₁₅, R₁₉, R_{20,} R₂₁, R₂₂ and R₂₃ is independently selected from H or deuterium; provided that there is at least one deuterium present in the compound. In other embodiments there are multiple deuterium atoms present in the compound.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of the compound of the invention. The concentration of deuterium, may be defined by the isotopic enrichment factor. The term “isotopic enrichment factor” as used herein means the ratio between the isotopic abundance and the natural abundance of a specified isotope. If a substituent in the compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into the compound of the invention include isotopes of hydrogen, other than deuterium, carbon, nitrogen, oxygen, and fluorine such as ³H, ¹¹C, ¹³C, ¹⁴C, ¹⁵N , ¹⁸F respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. The isotopically-labeled compounds can generally be prepared by conventional techniques known to those skilled in the art or by processes analogous to those described preparation of the compound of the invention by using an appropriate isotopically-labeled reagents in place of the non-labeled reagent previously employed.

As used herein, the terms “free form” or “free forms” refers to the compound in non-salt form, such as the base free form or the acid free form of a respective compound, e.g. the compounds specified herein (e.g. mavoglurant or further pharmaceutical active ingredient, for example, as defined herein).

As used herein, the terms “salt”, “salts” or “salt form” refers to an acid addition or base addition salt of a respective compound, e.g. the compounds specified herein (e.g. mavoglurant or further pharmaceutical active ingredient, for example, as defined herein). “Salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein (e.g. mavoglurant or further pharmaceutical active ingredient, for example, as defined herein), may be capable of forming acid and/or base salts by virtue of the presence of amino and/or carboxyl groups or groups similar thereto. The compound of the invention is capable of forming acid addition salts, thus, as used herein, the term pharmaceutically acceptable salt of mavoglurant means a pharmaceutically acceptable acid addition salt of mavoglurant.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

The compounds specified herein (e.g. mavoglurant or the further pharmaceutical active ingredient, for example, as defined herein) can be administered by conventional route, in particular orally, such as in the form of tablets, capsules, caplets or particulates, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in “Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5”. In particular, WO2014/199316 describes formulations comprising mavoglurant, in particular modified release formulations thereof, and is incorporated herein by reference, more particularly the Examples, the preferred embodiments and claims therein.

The pharmaceutical composition or combination of the present invention can be in a unit dosage form (e.g. tablet, capsule, caplet or particulate) comprising an amount ranging of from, for example, 1 mg to 300 mg, in particular of from 50 mg to 200 mg, such as 50 mg to 100 mg, more particularly 200 mg, of mavoglurant (referring to an amount of the free form of mavoglurant, and if a salt thereof is used the amount will be adapted accordingly; in particular mavoglurant is in the free form). For the above-mentioned uses/treatment methods the appropriate dosage may vary depending upon a variety of factors, such as, for example, the age, weight, sex, the route of administration or salt employed. In patients with, for example, of from 50-70 kg body weight, an indicated daily dosage is, for example, 200 mg/b.i.d (referring to an amount of the free form of mavoglurant, and if a salt thereof is used the amount will be adapted accordingly).

### ABBREVIATIONS

DSM-5=Diagnostic and Statistical Manual of Mental Disorders, 5th Ed.
AUD=Alcohol Use Disorder
TLFB=Timeline Follow-Back
mg=milligram
bid=b.i.d=twice (two times) a day
msec=millisecond
° C.=degree Celsius
cm=centimeter
FDA=Food and Drug Administration
MIDT=MID task=monetary incentive delay task
FHP=Alcohol use disorder (AUD) family history positive
FHN=Alcohol use disorder (AUD) family history negative
   The following Examples serve to illustrate the invention without limiting the scope thereof.

### EXAMPLES

The term “Compound (I)” or mavoglurant, as used in the context of these examples, refers to the free form.

### Example 1

### Trial Participants:

Alcohol use disorder (AUD) family history negative (FHN; n=7) and family history positive (FHP; n=5) individuals. As previously reported (Andrews M M, Meda S A, Thomas A D, Potenza M N, Krystal J H, Worhunsky P, Stevens M C, O'Malley S S, Book G A, Pearlson G D. Individuals Family History Positive for Alcoholism Show fMRI Abnormalities in Reward Sensitivity that are Related to Impulsivity Factors. Biol Psychiatry. 2011; 69:675-683), FHP status was determined by participants needing to have at least one parent with an AUD operationalized via the Family History Assessment Module (FHAM) developed by COGA, (https://cogastudy.org/phase-i-instruments-family-history-assessment-module-fham; https://www.niaa.nih.gov/research/major-initatives/collaborative-studies-genetics-alcoholism-coga-study) plus one or more other affected close (1st or 2nd degree) relatives. FHN had no affected close relatives.

### Trial Procedure:

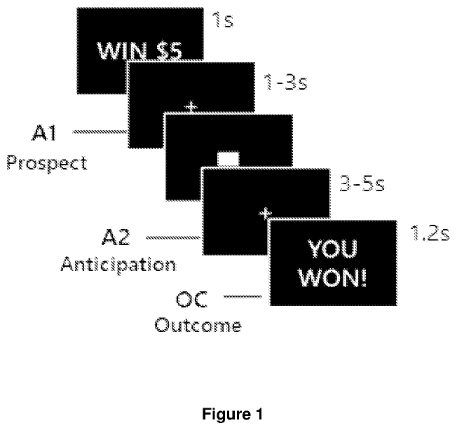

Mavoglurant or placebo was given in a double-blind, randomized fashion prior to administration of the fMRI MIDT task (see below), which permits modeling of two anticipatory phases, A1 or prospect and A2 or anticipation (Andrews M M, Meda S A, Thomas A D, Potenza M N, Krystal J H, Worhunsky P, Stevens M C, O'Malley S S, Book G A, Pearlson G D. Individuals Family History Positive for Alcoholism Show fMRI Abnormalities in Reward Sensitivity that are Related to Impulsivity Factors. Biol Psychiatry. 2011; 69:675-683; Jia Z, Worhunsky P D, Pearlson G D, Carroll K M, Rounsaville B J, Potenza M N. An initial study of neural responses to monetary incentives as related to treatment outcome in cocaine dependence. Biol Psychiatry. 2011; 70:553-560). fMRI data were acquired on Siemens Skyra 3T MRI scanner. Functional MID task data were acquired using a multiband gradient-echo sequence (Axial, TR=720 ms, TE=30 ms, FOV=240 mm, flip angle=60°, acquisition matrix=80×80, voxel size=3 mm³, number of slices=48, multi band factor=8, iPAT=1 ). In this between-subject, placebo-controlled fMRI study, participants received (one time) either 200 mg of mavoglurant [mavoglurant (free form) provided as modified release formulation (e.g. in WO2014/199316)] or placebo, 90 min prior to fMRI, and then conducted the MIDT. A nucleus accumbens (NAcc)/ventral striatal mask was generated, with coordinates and spatial locations obtained from Cerefy (Nowinski W L. The Cerefy brain atlases: continuous enhancement of the electronic talairach-tournoux brain atlas. Neuroinformatics. 2005; 3:293-300) and electronic Talairach-Tournoux brain atlases. This NAcc mask was further refined based on additional information (9, 14, 42-44) and edited using MARINA software (Walter B, Blecker C, Kirsch P, Sammer G, Schienle A, Stark R. MARINA: An easy to use tool for the creation of MAsks for Region of INterest Analyses. Research Gate. 2002; https://www.researchgate.net/publication/286632632). Anatomical location and spatial validity were verified by an imaging expert. This region of interest is described in our prior MIDT research (Patel K T, Stevens M C, Meda S A, Muska C, Thomas A D, Potenza M N, Pearlson G D. Robust Changes in Reward Circuitry during Reward Loss in Current and Former Cocaine Users during Performance of a Monetary Incentive Delay Task. Biol Psychiatry. 2013; 72:529-537) and is largely similar to other ventral striatal regions of interest from other atlases (https://fsl.fmrib.ox.ac.uk/fsl/fslwiki/Atlases).
In our version of the MIDT, participants complete one or two runs consisting of 55, 13-second trials. During trials, subjects see word cues (e.g., WIN $1, duration=1000 msec), fixate on a crosshair for a variable interval (delay=1000−3000 msec), respond with a button press to a target square that appears for a variable length of time, fixate on a crosshair for a variable interval (duration=1000−3000 msec), then receive feedback (1200 msec) notifying them whether or not they have won (or not lost) or not won (or lost) money during that trial (FIG. 1). Task difficulty, based on reaction times collected before scanning, is set such that each participant succeeds on approximately 67% of target responses, as was done in initial human studies [Knutson B, Fong G W, Adams C M, Varner J L, Hommer D. Dissociation of reward anticipation and outcome with event-related fMRI. Neuroreport. 2001; 12:3683-3687; Knutson B, Fong G W, Bennett S M, Adams C M, Hommer D. A region of mesial prefrontal cortex tracks monetarily rewarding outcomes: characterization with rapid event-related fMRI. Neuroimage. 2003; 18:263-272; Knutson B, Adams C M, Fong G W, Hommer D. Anticipation of increasing monetary reward selectively recruits nucleus accumbens. J Neurosci. 2001; 21:RC159 (151-155)]. Word cues specify potential reward, potential punishment, or neutral outcomes. Reward cues (winning $1 (n=11 ), or $5 (n=11 )), punishment cues (losing $1 (n=11 ), or $5 (n=11 )), and neutral cues (Win/Lose $0 (n=11 )) are included. Trial types are pseudo-randomly ordered. Subjects know that compensation is performance-based, as is detailed in our published studies (e.g. Balodis I M, Kober H, Worhunsky P D, Stevens M C, Pearlson G D, Potenza M N. Diminished fronto-striatal activity during processing of monetary rewards and losses in pathological gambling. Biol Psychiatry. 2012; 71:749-757; Andrews M M, Meda S A, Thomas A D, Potenza M N, Krystal J H, Worhunsky P, Stevens M C, O'Malley S S, Book G A, Pearlson G D. Individuals Family History Positive for Alcoholism Show fMRI Abnormalities in Reward Sensitivity that are Related to Impulsivity Factors. Biol Psychiatry. 2011; 69:675-683; Garrison K A, Yip S W, Balodis I M, Carroll K M, Potenza M N, Krishnan-Sarin S. Reward-related frontostriatal activity and smoking behavior among adolescents in treatment for smoking cessation. Drug Alcohol Depend. 2017; 177:268-276; Jia Z, Worhunsky P D, Pearlson G D, Carroll K M, Rounsaville B J, Potenza M N. An initial study of neural responses to monetary incentives as related to treatment outcome in cocaine dependence. Biol Psychiatry. 2011; 70:553-560; Patel K T, Stevens M C, Meda S A, Muska C, Thomas A D, Potenza M N, Pearlson G D. Robust Changes in Reward Circuitry during Reward Loss in Current and Former Cocaine Users during Performance of a Monetary Incentive Delay Task. Biol Psychiatry. 2013; 72:529-537; Balodis I M, Grilo C M, Kober H, Worhunsky P D, White M A, Stevens M C, Pearlson G D, Potenza M N. A pilot study linking reduced fronto-Striatal recruitment during reward processing to persistent bingeing following treatment for binge-eating disorder. The International journal of eating disorders. 2014; 47:376-384; Balodis I M, Kober H, Worhunsky P D, White M A, Stevens M C, Pearlson G D, Sinha R, Grilo C M, Potenza M N. Monetary reward processing in obese individuals with and without binge eating disorder. Biological Psychiatry. 2013; 73:877-886; Balodis I M, Kober H, Worhunsky P D, Stevens M C, Pearlson G D, Carroll K M, Potenza M N. Neurofunctional reward processing changes in cocaine dependence during recovery. Neuropsychopharmacol. 2016; 41:2112-2121; Lichenstein S D, Scheinost D, Potenza M N, Carroll K M, Yip S W. Dissociable neural substrates of opioid and cocaine use identified via connectome-based modelling. Mol Psychiatry. in press; Yip S W, Scheinost D, Potenza M N, Carroll K M. Connectome-Based Prediction of Cocaine Abstinence. Am J Psychiatry. 2019; 176:156-164; Yip S W, DeVito E E, Kober H, Worhunsky P D, Carroll K M, Potenza M N. Anticipatory reward processing among cocaine-dependent individuals with and without concurrent methadone-maintenance treatment: Relationship to treatment response. Drug Alcohol Depend. 2016; 166:134-142; Yip S W, DeVito E E, Kober H, Carroll K M, Potenza M N. Pretreatment measures of brain structure and reward-processing brain function in cannabis dependence: An exploratory study of relationships with abstinence during behavioral treatment. Drug Alcohol Depend. 2014; 140:33-41 ).

### Results

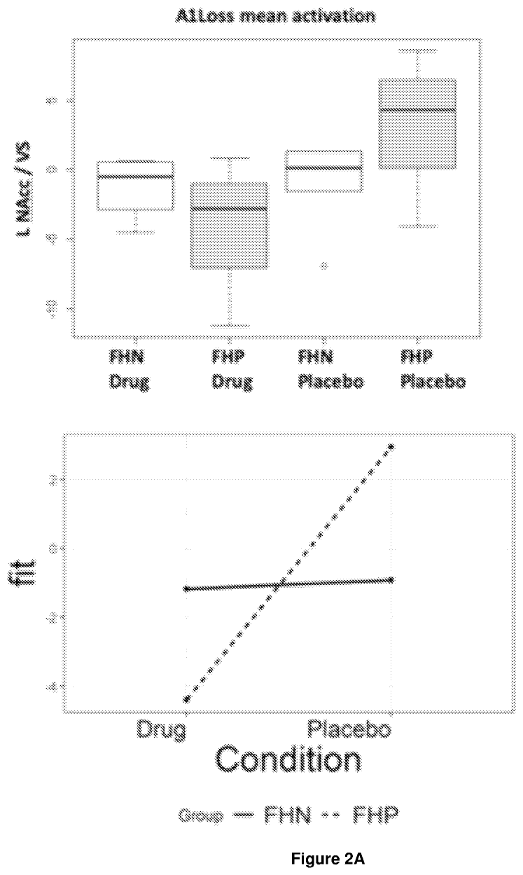

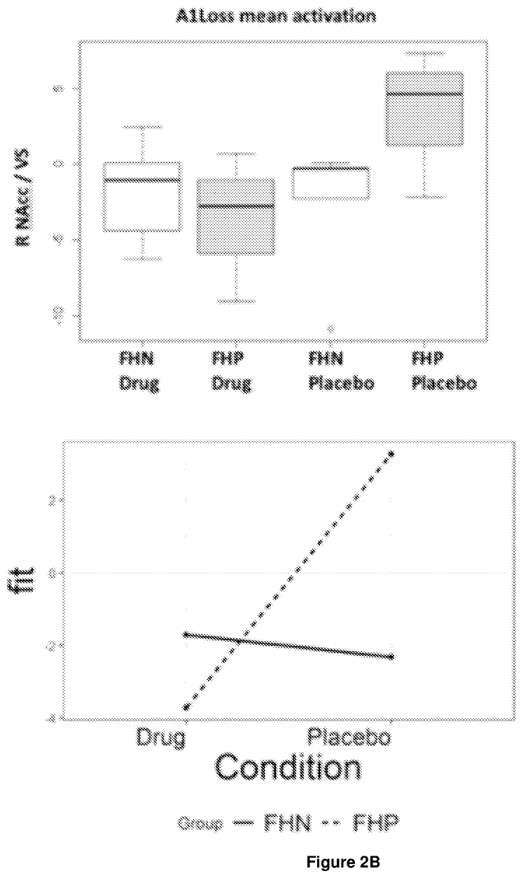

FIGS. 2A and 2B depicts plots from A1 phase of the MIDT, response to losses, in right (R) and left (L) nucleus accumbens (NAcc)/ventral striatum (VS). In the placebo phase, FHP individuals generate a larger signal than FHN individuals bilaterally. As seen in bar graphs, this difference is reduced by mavoglurant in FHP individuals to levels equivalent to or below those of FHN individuals (FIGS. 2A and 2B). The FHN signal in comparison is essentially unresponsive to drug. This interaction is significant at p<0.001, uncorrected. The data obtained suggest that mavoglurant produces opposing effects on the neural response during fMRI in brain regions implicated in reward and addiction, increasing neural response in the ventral striatum during the A1 phase of processing in FHN individuals and attenuating it in FHP individuals. The findings indicate at p<0.001 (see above) that mavoglurant appears to “normalize” circuit activity in FHP individuals, making their neural activity look similar to FHN individuals on placebo.

### CONCLUSIONS

Alcohol use disorder (AUD) family history positive (FHP) individuals show differences in the A1 and A2 phases of reward processing (Andrews M M, Meda S A, Thomas A D, Potenza M N, Krystal J H, Worhunsky P, Stevens M C, O'Malley S S, Book G A, Pearlson G D. Individuals Family History Positive for Alcoholism Show fMRI Abnormalities in Reward Sensitivity that are Related to Impulsivity Factors. Biol Psychiatry. 2011; 69:675-683), in which relatively increased activations are seen in reward-related brain regions in individuals with or at increased risk for addiction. Similar relationships have been found between decision-making measures and ventral striatal activations during the A1 (prospect) phases of working for reward and to avoid loss in people with and without gambling disorder (Balodis I M, Linnet J, Arshad F, Worhunsky P D, Stevens M C, Pearlson G D, Potenza M N. Relating neural processing of reward and loss prospect to risky decision-making in individuals with and without gambling disorder. International Gambling Studies. 2018; 18:269-285). The data from our study above indicate that mavoglurant effects the A1 phase of the monetary incentive delay task (MIDT) in individuals with a positive family history of alcoholism (FHP). These results support thus the potential therapeutic impact of mavoglurant in individuals with gambling disorder or gaming disorder.

1. A method for the treatment of gambling disorder;
for the treatment of symptoms of depression or anxiety associated with gambling disorder;
in a treatment for the reduction of gambling by a gambling disorder patient;
in a treatment to prevent relapse into gambling by a gambling disorder patient; and/or
in a treatment to promote gambling abstinence by a gambling disorder patient;
comprising administering to a subject in need thereof an effective amount of mavoglurant, or a pharmaceutically acceptable salt thereof.

2. The method according to claim 1, which is for the treatment of the symptoms of depression or anxiety associated with gambling disorder.

3. The method according to claim 1, which is in a treatment for the reduction of gambling by a gambling disorder patient.

4. The method according to claim 1, which is in a treatment to prevent relapse into gambling by a gambling disorder patient.

5. The method according to claim 1, which is in a treatment to promote gambling abstinence by a gambling disorder patient.

6. The method according to claim 1, wherein gambling disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder, in particular depression or anxiety.

7. The method according to claim 1, wherein the gambling disorder is comorbid with substance use disorder (e.g. cocaine use disorder, alcohol use disorder, opioid use disorder or amphetamine-type stimulant use disorder).

8. The method according to claim 1, which is combined with standardized psychological treatment, for example, at individual or group level.

9. The method according to claim 1, which is combined with psychosocial or behavioral therapy or combination thereof, in particular contingency management based therapy.

10. The method according to claim 9, wherein the psychosocial or the behavioral therapy is computer-assisted.

11. The method according to claim 1, wherein mavoglurant, or a pharmaceutically acceptable salt thereof, is administered in combination with a further active agent, for example wherein the further active agent is an antidepressant or an anxiolytic.

12. The method according to claim 1, wherein the patient has a genetic variation associated with a substance use disorder.

13. The method according to claim 1, wherein mavoglurant, or a pharmaceutically acceptable salt thereof, is administered in an immediate-release form or a modified-release form.

14. The method according to claim 1, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.

15. The method according claim 1, wherein gambling disorder is associated with binge drinking or alcohol use disorder.

16. The method according to claim 1, wherein the gambling disorder is mild gambling disorder, moderate gambling disorder or severe gambling disorder.

17. The method according to claim 1, wherein the gambling disorder is episodic or persistent.

18. The method according to claim 1, wherein the gambling disorder is in early remission or in sustained remission.

19. The method according to claim 1, wherein gambling is replaced by gaming.

20. A method for the treatment of gaming disorder;
for the treatment of the symptoms of depression or anxiety associated with gaming disorder;
in a treatment for the reduction of gaming by a gaming disorder patient;
in a treatment to prevent relapse into gaming by a gaming disorder patient; and/or
in a treatment to promote gaming abstinence by a gaming disorder patient;
comprising administering to a subject in need thereof an effective amount of mavoglurant, or a pharmaceutically acceptable salt thereof.

21. The method according to claim 1, which is for the treatment of the symptoms of depression or anxiety associated with gaming disorder.

22. The method according to claim 1, which is in a treatment for the reduction of gaming by a gaming disorder patient.

23. The method according to claim 1, which is in a treatment to prevent relapse into gaming by a gaming disorder patient.

24. The method according to claim 1, which is in a treatment to promote gaming abstinence by a gaming disorder patient.
